(19) United States
(12) Patent Application Publication
(10) Pub. No.: US 2003/0088253 A1
(43) Pub. Date: May 8, 2003

(54) Dual action ophthalmic implant extractor

(76) Inventor: Randolph L. Seil, House Springs, MO (US)

Correspondence Address: RITA D. VACCA, BAUSCH & LOMB, INC., ONE BAUSCH & LOMB PLACE, ROCHESTER, NY, 14604-2701, US

(21) Appl. No.: 10/045,291

(22) Filed: Nov. 7, 2001

(51) Int. Cl. A61F 9/00

(52) U.S. Cl. 606/107

(57) ABSTRACT

A surgical dual action ophthalmic implant extractor device useful for ophthalmic implant bisection and/or explantation through a relatively small incision in an eye. The surgical device generally comprises a head portion with a jaw portion and a cutter portion and a removably attached handle portion with a spring biased actuator in communication with the jaw portion and cutter portion for activation thereof.

### FIELD OF THE INVENTION

The present invention relates to a surgical device useful for the extraction of ophthalmic implants and a method of making and using the same. More particularly, the present invention relates to a dual action intraocular lens extractor that enables a surgeon to hold and bisect an intraocular lens with one hand motion.

### BACKGROUND OF THE INVENTION

The natural crystalline lens of the eye plays a primary role in focusing light onto the retina for proper vision. However, vision through the natural lens may become impaired due to an injury or the formation of a cataract caused by aging or disease. To restore vision in the case of a cataractous lens, the natural lens is typically removed and replaced with an artificial lens. Implantation of an artificial lens may also be useful to make a refractive correction in an eye without the removal of a non-cataractous natural crystalline lens.

Many surgical procedures have been developed for removing the natural lens if cataractous. Typically, a slender implement is inserted through a small incision in the eye to contact the natural lens. The implement includes a cutting edge that is ultrasonically vibrated to emulsify the lens. The emulsified fragments of the lens are then aspirated out of the eye through a passage located in the proximity of the cutting edge. The slender nature of the implement enables extraction of the lens through a small incision in the eye. Removal of the natural lens through a relatively small incision is preferred over other procedures requiring larger incisions. Procedures requiring a relatively smaller incision can lessen the trauma and complications experienced both during surgery and postoperatively.

Because the incision required to remove a natural lens is relatively small, intraocular lenses (IOLs) that do not require any enlargement of the surgical incision for implantation are preferred. IOLs commonly include a generally disk shaped optic which focuses light on the retina and at least one outwardly extending haptic portion for proper positioning and support of the optic within the eye. Flexible IOLs enable a lens to be folded and compressed so as to occupy a smaller cross-sectional area for passage of the IOL through a relatively small surgical incision. Such IOLs may be implanted in aphakic eyes, wherein the natural lens has been removed, or alternatively, in phakic eyes, wherein the natural lens has not been removed.

Commercially successful IOLs have been made from a variety of biocompatible materials, ranging from more rigid materials such as polymethyl methacrylate (PMMA) to softer, more flexible materials capable of being folded or compressed such as silicones and certain acrylics. Haptic portions of the IOLs have been formed separately from the optic portion and later connected thereto through processes such as heat, physical staking and/or chemical bonding. IOLs with haptics so attached are commonly referred to as “multi-piece” IOLs. IOLs are also commonly produced with haptics formed as an integral part of the optic portion in what is commonly referred to as “single-piece” IOLs.

Softer, more flexible IOLs have gained in popularity in recent years due to their ability to be compressed, folded, rolled or otherwise deformed. Such softer IOLs may be deformed prior to insertion thereof through an incision in the cornea of an eye. Following insertion of the IOL in an eye, the IOL returns to its original pre-deformed shape due to the memory characteristics of the soft material.

Softer, more flexible IOLs as just described may be implanted into an eye through an incision that is much smaller, i.e., 2.8 to 3.5 mm, than that necessary for more rigid IOLs, i.e., 4.8 to 6.0 mm. A larger incision is necessary for more rigid IOLs because the lens must be inserted through an incision in the cornea slightly larger than that of the diameter of the inflexible IOL optic portion. Accordingly, more rigid IOLs have become less popular in the market since larger incisions have been found to be associated with an increased incidence of postoperative complications, such as induced astigmatism.

Although IOL implants provide significant benefits to most recipients following cataractous lens removal, or simply for refractive correction, this is not always the case. It is estimated that up to fifty percent (50%) of all patients who have IOL implants placed within the lens capsule of the eye later develop posterior capsular opacification (PCO) or secondary cataracts within five years following surgery. PCO is an opacification of IOL implants caused by the deposit of cells and fibers on the posterior surface of the IOL implant and the posterior capsular membrane. These deposits of cells and fibers obstruct light passing through the IOL implant and obscure the patient's vision. Other IOL related complications may include but are not limited to the formation of “glistenings” or water pockets within an IOL, IOL manufacturer recalls and changes over time in the degree of refractive correction required by the IOL recipient.

Commonly, in the case of an IOL complication, it becomes necessary for the IOL implant to be explanted. Because of the noted shortcomings of past and current IOL extractor products, there is a need for a surgical IOL extractor device designed to remove IOL implants with surgical ease and minimal tissue trauma.

### SUMMARY OF THE INVENTION

The present invention pertains to a dual action intraocular lens (IOL) extractor and a method of making and using the same to explant or remove an IOL from within an eye. The dual action IOL extractor of the present invention in its preferred construction provides a sterilizable and reusable surgical IOL explantation device with a movable jaw and cutter assembly. In using the subject dual action IOL extractor, the movable jaw and cutter assembly is inserted through a relatively small incision within an eye thus allowing the same to contact the IOL implant to be explanted. The jaw assembly is positioned with an anterior jaw member in contact with an anterior surface of the IOL and a posterior jaw member in contact with a posterior surface of the IOL. Upon actuation of a cutter member, the jaw assembly grasps the IOL and the cutter member bisects the IOL allowing for small incision removal thereof from the eye.

Accordingly, it is an object of the present invention to provide a surgical device for intraocular lens explantation through a relatively small incision.

Another object of the present invention is to provide a dual action surgical device for holding and cutting an intraocular lens.

Another object of the present invention is to provide a surgical device for intraocular lens explantation capable of sterilization and reuse.

Another object of the present invention is to provide a surgical device for intraocular lens explantation that minimizes tissue trauma.

Another object of the present invention is to provide a surgical device for intraocular lens explantation that is relatively simple and convenient to use in a surgical procedure.

Still another object of the present invention is to provide a surgical device for intraocular lens explantation that is reliable and cost effective.

These and other objectives and advantages of the present invention, some of which are specifically described and others that are not, will become apparent from the detailed description, examples and claims that follow.

### BRIEF DESCRIPTION OF THE DRAWINGS

FIG. 1 is a side plan view of a dual action ophthalmic implant extractor made in accordance with the present invention;

FIG. 2 is a side plan view of a head portion of the extractor of FIG. 1 rotated 180 degrees;

FIG. 3 is a plan view of a base portion of the head portion of FIG. 2;

FIG. 4 is a plan view of a connection portion of the handle portion of FIG. 10;

FIG. 5 is side plan view of a jaw portion of the extractor of FIG. 1;

FIG. 6 is a side plan view of a jaw portion and a cutter portion of the extractor of FIG. 1;

FIG. 7 is a bottom plan view of a lock block;

FIG. 8 is a side plan view of the jaw portion and cutter portion of FIG. 6 rotated 180 with the lock block of FIG. 7 rotated 90 degrees;

FIG. 9 is a cross-sectional plan view of the jaw portion, cutter portion and lock block of FIG. 8;

FIG. 10 is a side plan view of a handle portion of the extractor of FIG. 1 with an extended actuator;

FIG. 11 is a cross-sectional plan view of the handle portion of FIG. 10; and

FIG. 12 is a side plan view of the extractor of FIG. 1 with an activated actuator and a jaw portion grasping a bisected ophthalmic implant.

### DETAILED DESCRIPTION OF THE INVENTION

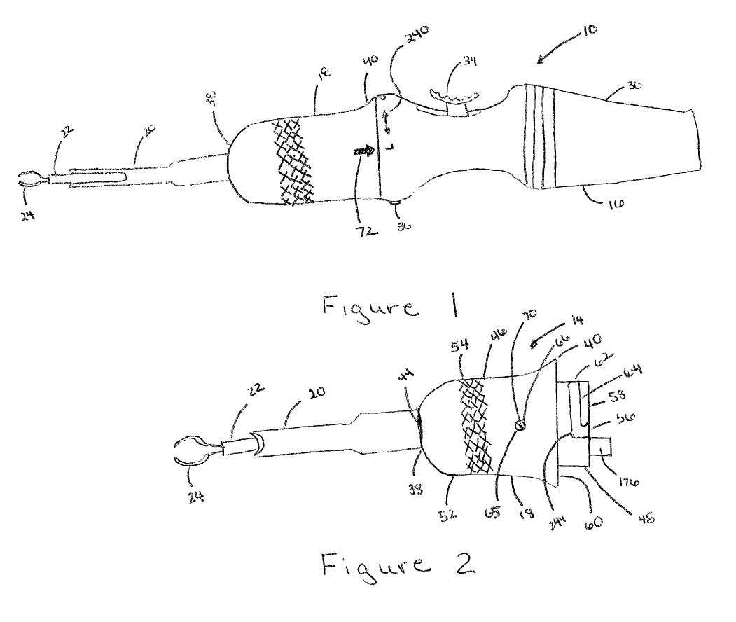

The present invention pertains to an ophthalmic surgical device, and more specifically, a dual action ophthalmic implant extractor 10 as illustrated in FIG. 1. Extractor 10 is preferably used to extract or explant ophthalmic implants such as for example but not limited to intraocular lenses (IOLs), capsular bag extension rings and the like from eyes. Extractor 10 enables a surgeon to both grasp and bisect an implanted ophthalmic device such as an IOL 12 through one hand action or movement for removal of IOL 12 through a relatively small incision.

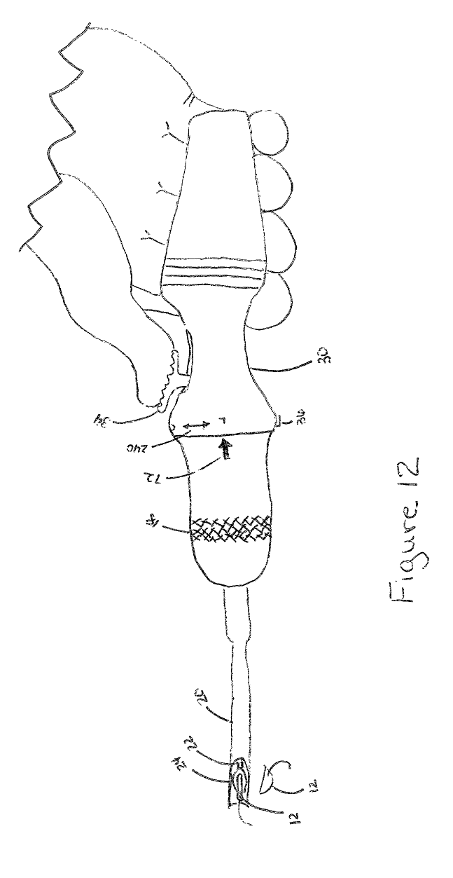

Extractor 10 made in accordance with the present invention generally comprises a head portion 14 as illustrated in FIG. 2, which interlocks with a handle portion 16 as illustrated in FIG. 10. Head portion 14 generally comprises housing member 18, cutter portion 20 with holder tube 22 and head spring 28, jaw portion 24 and lock block 26. Handle portion 16 generally comprises base housing 30, base spring 32, actuator 34 and lock screw 36.

Housing member 18 of head portion 14 is generally tubular in shape with opposed exterior end 38 and interior end 40 fluidly connected by bore 42. Exterior end 38 with exterior opening 44 of bore 42 is preferably inwardly tapered so as to be of smaller diameter than that of mid-section 46 of housing member 18. Conversely, interior end 40 of housing member 18 is preferably outwardly tapered or flared so as to be of greater diameter than that of mid-section 46 of housing member 18. Interior end 40 is likewise of a greater diameter than that of extended flange 48 thereby forming abutment edge 60. Extended flange 48 with interior opening 50 of bore 42 has receiving channel 56 that extends from free edge 58 of extended flange 48 to approximately midpoint 62 between free edge 58 and abutment edge 60. At midpoint 62, receiving channel 56 fluidly connects with locking channel 64, which extends from receiving channel 56 in a clockwise direction parallel between free edge 58 and abutment edge 60 for approximately 90 degrees around extended flange 48. In alignment with receiving channel 56 in interior end 40 is aperture 65. Interior edge 66 of aperture 65 preferably has threading (not shown) for purposes of nonpermanent engagement with threaded stabilizing screw 70. Etched on exterior surface 52 of housing member 18 opposite aperture 65 and stabilizing screw 70 is indicia 72 useful in interlocking head portion 14 with handle portion 16. Additionally, preferably at approximately mid-section 46 of housing member 18 on exterior surface 52, textured zone 54 circumscribes housing member 18. Alternatively, all or any desired portion or portions of exterior surface 52 may form textured zone 54.

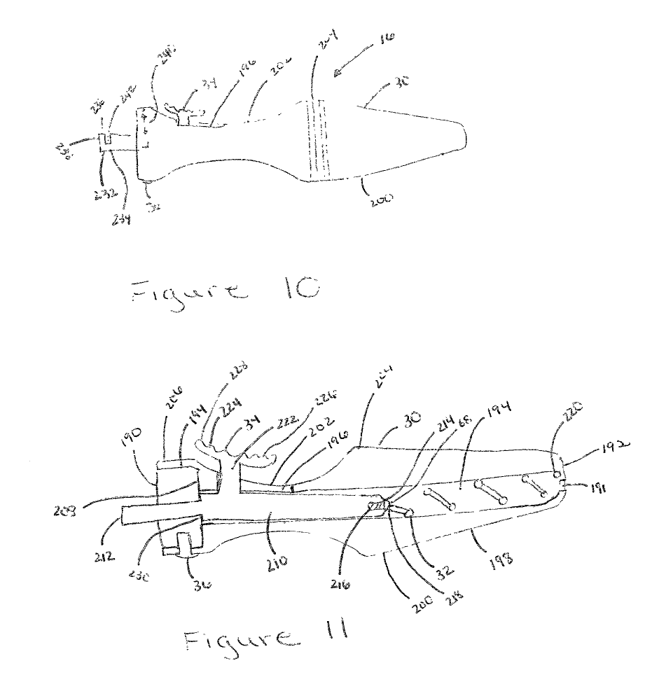

Cutter portion 20 as best illustrated in FIGS. 6 and 8 sized to extend through bore 42 of housing member 18 comprises base 74, cutter tube 76, holder tube 22 and head spring 28. Base 74 is generally cylindrical in shape having a diameter sized to be accepted and generally maintained within bore 42. Base 74 has opposed interior edge 78 and exterior edge 174. Permanently attached to or integrally formed with exterior edge 174 is peg 176. Peg 176 extends from posterior quadrant 178 of exterior edge 174 as best illustrated in FIG. 3. Extending from peg 176 to the adjacent posterior quadrant 180 is tooth member 182. Permanently attached to or integrally formed with interior edge 78 of base 74 is cutter tube 76. Cutter tube 76 has an interior tube edge 80 that defines attached tube opening 82. Opposite interior tube edge 80 is free end 84. Attached tube opening 82 and free end 84 are fluidly connected by means of cutter bore 86. Cutter tube 76 at interior tube edge 80 is sized to be of a diameter acceptable for passage through exterior opening 44. Interior portion 88 of cutter tube 76 is positioned within housing member 18 so that exterior portion 90 extends outwardly from exterior opening 44 outside of housing member 18. Extending a distance from interior tube edge 80 is spring region 92. Extending from spring region 92 is blade portion 94. Blade portion 94 is of a smaller diameter than that of spring region 92. Likewise, the diameter of cutter bore 86 coincides with that of spring region 92 and blade portion 94. At free end 84 of blade portion 94, half of cutter tube 76 is removed to form elongated opening 96. The remaining cutter tube 76 at free end 84 is sharpened to form crescent blade 98. Maintained within cutter bore 86 of spring region 92, between base 74 and blade portion 94 is head spring 28.

Holder tube 22, with the exception of flange 100, is of a diameter acceptable within blade portion 94. Holder tube 22 is preferably of uniform diameter throughout the length thereof, with the exception of the enlarged diameter of flange 100 that extends outwardly from interior end 102 of holder tube 22. Flange 100 has a diameter acceptable within cutter bore 86 of spring region 92 but is too large for passage or placement within cutter bore 86 of blade portion 94 as best illustrated in FIG. 9. Interior end 102 defines interior opening 104 of holder tube 22 and is opposed to exterior end 106 that defines free opening 108. Interior opening 104 and free opening 108 are fluidly connected by means of holder bore 110. At least a portion of holder tube 22 extends outwardly from free end 84 due to an outwardly directed force on flange 100 by head spring 28.

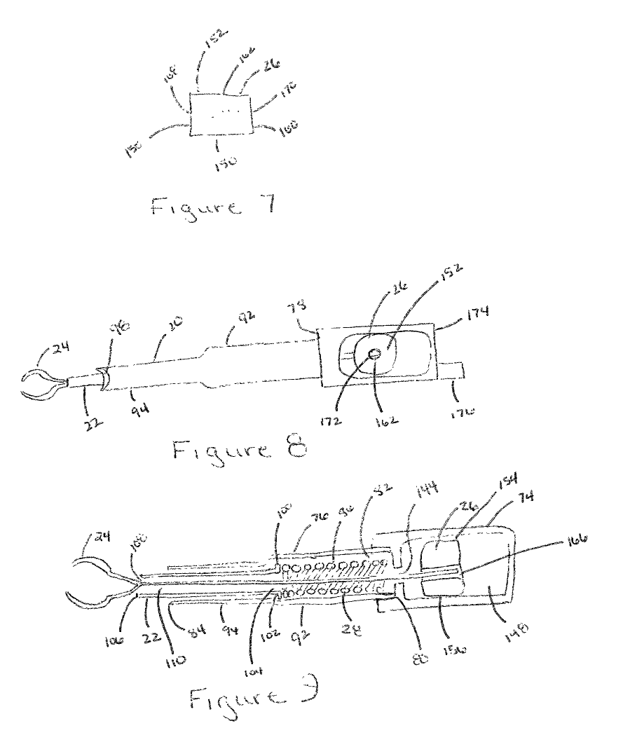

Jaw portion 24 as best illustrated in FIG. 5, comprises an anterior arced member 112, a posterior arced member 114, outwardly biased means 116 and shaft 118. Anterior arced member 112 has a convex exterior surface 120, a concave interior surface 122, a free tip 128 and an opposed attached end 130. Posterior arced member 114 has a convex exterior surface 124, a concave interior surface 126, a free tip 132 and an opposed attached end 134. Attached ends 130 and 134 are permanently connected to or integrally formed with tip 140 of anterior leg 136 and tip 142 of posterior leg 138, respectively, of outwardly biased means 116. Opposite tip 140 and 142, anterior leg 136 and posterior leg 138 join to form shaft 118. Shaft 118 is generally cylindrical with a diameter of a size acceptable for placement within holder bore 110, cutter bore 86 and base bore 144. Free end 146 of shaft 118 extends into elongated chamber 148 of base 74 by means of base bore 144.

Lock block 26 as best illustrated in FIGS. 7 through 9, is generally a rectangular block with opposed first free side 150 and second free side 152, opposed anterior sliding side 154 and posterior sliding side 156, and opposed attachment side 158 and abutting side 160. Shaft bore 166 passes from block opening 168 of attachment side 158 to block opening 170 of abutting side 160 creating a fluid connection therebetween. Shaft bore 166 is of a diameter suitable for receiving shaft 118 therein. In the middle of free side 152 is aperture 162. Interior edge 172 of aperture 162 sized to accept stabilizing screw 70 so as to allow stabilizing screw 70 to contact shaft 118 for maintaining shaft 118 within shaft bore 166. Thus stabilizing lock block 26 and jaw portion 24 are non-movably secured within housing member 18 through stabilizing screw 70, while base 74, cutter portion 20 and holder tube 22 remain mobile within housing member 18. Base 74, cutter portion 20 and holder tube 22 remain mobile within housing member 18 through elongated chamber 148, which is sized to enable base 74 to slide around stationary lock block 26.

Handle portion 16 includes base housing 30, base spring 32, actuator 34 and lock screw 36. Base housing 30 is preferably essentially tubular in shape with an open end 190 and opposed free end 192. Extending for a defined distance within base housing 30 from open end 190 is interior bore 194. In fluid communication with interior bore 194 is actuator slot 196. The exterior 198 of base housing 30 preferably varies in diameter for comfortable handling. One such embodiment of base housing 30 as best illustrated in FIGS. 10 and 11 provides for a base mid portion 200 of a particular diameter. The diameter of base housing 30 gradually decreases from the point of base mid portion 200 in the direction of free end 192. Adjacent base mid portion 200 opposite free end 192 is actuator region 202 of a diameter also less than that of base mid portion 200. On base mid portion 200 near actuator region 202 is preferably texturing 204 to decrease handling slippage. However, it is contemplated by the present invention that all or any portion(s) of exterior 198 may have texturing 204 for ease in handling. Adjacent actuator region 202 at open end 190 is attachment region 206 of relatively the same diameter as that of base mid portion 200. On exterior 198 at open end 190 is indicia 240 as best illustrated in FIG. 10. Indicia 240 preferably includes an “O” to indicate “open” in alignment with actuator slot 196. When looking into open end 190, 90 degrees clockwise from “O” is “L” to indicate “locked”. Between “O” and “L”, indicia 240 includes a double-headed arrow.

From open end 190 within attachment region 206 of base housing 30, interior bore 194 has a diameter sized to accommodate extended flange 48 of housing member 18. Within actuator region 202 and base portion 200, internal bore 194 has a uniform diameter smaller than that of the uniform diameter thereof within attachment region 206. The change in the diameter of interior bore 194 from attachment region 206 to actuator region 202, forms abutment 208.

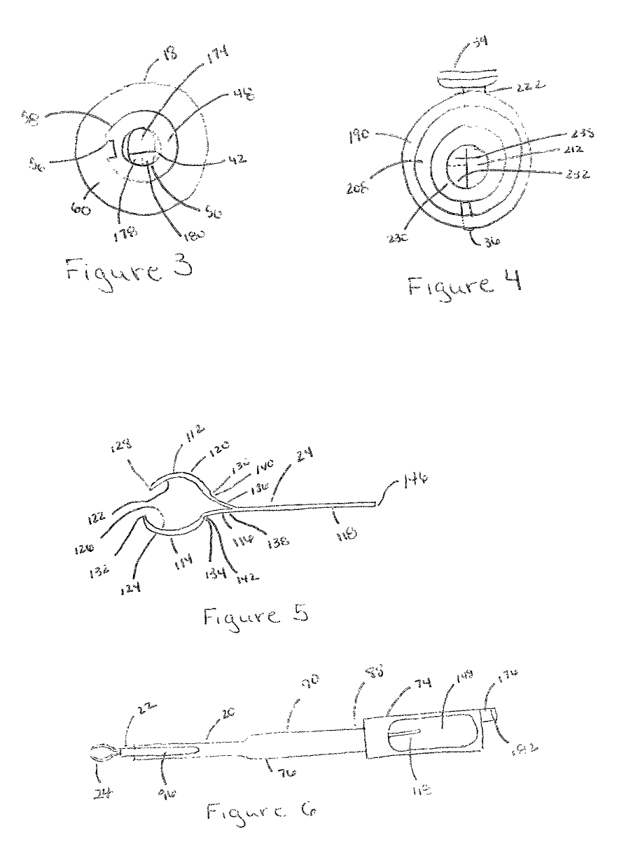

Actuator 34 is generally cylindrical having a body portion 210 and opposed attachment end 212 and spring end 214. Body portion 210 is generally of a uniform diameter sized to be accepted within interior bore 194. However, for a defined length, a portion of body portion 210 at attachment end 212 is of a smaller diameter coinciding with the diameter of exterior edge 174. The change in the diameter of body portion 210 at attachment end 212 forms abutment 230. Permanently attached to or integrally formed with attachment end 212 is peg 232. Peg 232 extends from posterior quadrant 234 of attachment end 212 as best illustrated in FIGS. 4 and 10. Extending from peg 232 to the adjacent anterior quadrant 236 is tooth member 238. In spring end 214 of body portion 210 is groove 216 to which first end 218 of spring 32 is attached by means of threaded screw 68. However, other methods of securely fixing spring 32 to actuator 34 would be acceptable such as through the use of a snap clamp or the like. Opposed second end 220 of spring 32 is securely attached within interior bore 194 at closed end 192 preferably by press friction fit or through the use of a threaded screw. Additionally, preferably located through closed end 192 is vent hole 191. Extending outwardly from body portion 210 of actuator 34 through actuator slot 196 beyond exterior 198 is actuator base 222. Integrally formed with actuator base 222 or securely attached thereto opposite body portion 210 is thumb button 224. The anterior surface 226 of thumb button 224 preferably has texturing 228 to avoid thumb slippage across anterior surface 226. Upon applying force to thumb button 224, actuator base 222 moves within actuator slot 196 toward open end 190 thereby causing body portion 210 of actuator 34 to extend beyond open end 190 and spring 32 to be extended. When actuator 34 is in its most extended position, abutment 208 and abutment 230 are in alignment.

In securely attaching head portion 14 to handle portion 16, indicia 72 on head portion 14 is aligned with “O” indicia 240 on handle portion 16 with abutment edge 60 in direct contact with open end 190. Head portion 14 is then turned 90 degrees with respect to handle portion 16 whereby indicia 72 on head portion 14 is aligned with “L” indicia 240 on handle portion 16 thereby locking head portion 14 onto handle portion 16. The mechanism whereby head portion 14 is locked onto handle portion 16 is explained as follows. When abutment edge 60 is placed in contact with open end 190, peg 176 and tooth 182 are in contact with attachment end 212 of actuator 34. Likewise, when abutment edge 60 is placed in contact with open end 190, peg 232 and tooth member 238 are in contact with exterior edge 174. At this point, peg 176 and peg 232 are positioned side by side extending from opposed posterior quadrants 178 and 234 respectively. When head portion 14 and handle portion are so positioned, lock screw 36 is positioned at innermost point 244 of receiving channel 56. Upon a 90 degree rotation of head portion 14 with respect to handle portion 16, whereby indicia 72 is aligned with “L” indicia 240, tooth 182 is positioned in space 242 between tooth member 238 and attachment end 212, and lock screw 36 slides 90 degrees within locking channel 64.

Once head portion 14 is securely locked onto handle portion 16, extractor 10 of the present invention is ready for use. In using extractor 10, a surgeon's fingers are preferably wrapped around base housing 30 allowing the thumb to rest on thumb button 224. A force may then be applied by the thumb to thumb button 224 causing spring 32 to become extended and causing actuator 34 to extend beyond open end 190 into bore 42. Upon actuator 34 extending into bore 42, base 74 is moved toward exterior end 38 of housing member 18 thus causing holder tube and cutter tube to extend further beyond exterior end 38. Since lock block 26 and jaw portion 24 by means of shaft 118 are held stationary within housing member 18 by stabilizing screw 70, when base 74 is moved toward exterior end 38 of housing member 18, lock block 26 is repositioned within elongated channel 148 of base 74 from a position in closest proximity to head spring 28 to a position of farthest proximity from head spring 28. As lock block 26 is repositioned, exterior end 106 of holder tube 22 slides over outwardly biased means 116 of jaw portion 24 to bring anterior leg 136 and posterior leg 138 into direct contact thus bringing free tip 128 into direct contact with free tip 132.. The movement of holder tube 22 over jaw portion 24 is stopped upon abutment of exterior end 106 with anterior arced member 112 and posterior arced member 114 since the same are not sized to be accepted within the diameter of holder tube 22. Although holder tube 22 stopped by arced members 112 and 114, cutter portion 20 continues to slide over holder tube 22 with the compression of head spring 28 until crescent blade extends outwardly beyond free tips 128 and 132 of jaw portion 24.

As illustrated in FIG. 12, in using extractor 10 of the present invention in a surgical procedure to explant IOL 12, an incision is made in the cornea of an eye. Extractor 10 is partially inserted within the surgical incision so as to allow anterior arced member 112 to slide over the anterior surface of IOL 12 and posterior arced member 114 to slide over the posterior surface of IOL 12 to be explanted. A force is applied by the surgeon's thumb to thumb button 224 causing actuator 34 to move forward within actuator slot 196 and free tips 128 and 132 of jaw portion 24 to clasp IOL 12. While IOL 12 is clasped between free tips 128 and 132, crescent blade 98 of cutter tube 76 continues to move past jaw portion 24 bisecting IOL 12. The portion of IOL 12 clasped between free tips 128 and 132 is then removed from the eye via the incision made in the cornea. Extractor 10 is then reinserted within the incision to grasp the remaining portion of IOL 12. The remaining portion of IOL 12 is grasped by again applying a force to thumb button 224, but not enough force to bisect the remaining portion of IOL 12 within the eye. The remaining portion of IOL 12 is then likewise removed from the eye via the corneal incision. Upon release of thumb button 224, the compression of head spring 28 is released as is the extension of base spring 32 so as to return extractor 10 to its original at rest locked position.

The surgical extractor device 10 of the present invention may be made from any suitable materials capable of reliable durability and preferably repeated sterilization in accordance with methods known by those skilled in the art of multiple-use surgical instrumentation applications. The preferred material for use in the manufacture of the subject surgical extractor device 10 is surgical stainless steel.

The above detailed description discloses the preferred embodiments of the present invention. Various other embodiments as well as many changes and alterations may be made without departing from the spirit and broader aspects of the invention as defined in the appended claims.

I claim:

1. An ophthalmic implant extractor surgical device comprising:
a jaw portion with arced members for grasping an ophthalmic implant;
a cutter portion capable upon activation of moving arced members into closer proximity with respect to one another and bisecting said ophthalmic implant while grasped between said arced members;
a handle portion removably attached to said jaw and cutter portions; and
an actuator portion within said handle portion in communication with said jaw portion and cutter portion for activation thereof from said handle portion.

2. The surgical device of claim 1 wherein said jaw portion includes an anterior arced member and a posterior arced member.

3. The surgical device of claim 1 wherein said jaw portion includes outwardly biased means attached to an anterior arced member and a posterior arced member.

4. The surgical device of claim 1 wherein said cutter portion includes a holder tube and a cutter tube formed with a blade opposite an elongated slot opening.

5. A method of making the surgical device of claim 1 comprising:
assembling a head portion to have a stationary jaw portion, and movable holder tube and cutter tube with respect thereto;
assembling a handle portion to have a spring biased actuator; and
removably attaching said head portion to said handle portion whereby said holder tube and cutter tube are in communication with and activated through said actuator.

6. A method of using the surgical device of claim 1 comprising:
inserting a portion of said jaw portion and said cutter portion into an incision in an eye with an implant; and
applying sufficient force to said actuator portion for said jaw portion to grasp said implant and for said cutter portion to bisect said implant.

7. A method of using the surgical device of claim 1 comprising:
inserting a portion of said jaw portion and said cutter portion into an incision in an eye with an implant; and
applying sufficient force to said actuator portion for said jaw portion to grasp said implant for removal thereof through said incision.

8. The method of claim 6 or 7 wherein said implant is an intraocular lens.

9. The method of claim 6 or 7 wherein said implant is a capsular bag extension ring.

10. The surgical device of claim 1, 2 or 3 wherein said implant is an intraocular lens implant or a capsular bag extension ring.
